SEQUENCE LISTING (3 sequences)

SEQ ID NO: 1 - DNA, 6117 bp, Homo sapiens
atgggggcctcttctccaagaagcccggagcctgtcgggccgccggcgcccggtctccccttctgctgcggaggatccctgctggcggttgtggtgctgcttgcgctgccggtggcctggggtcaatgcaatgccccagaatggcttccatttgccaggcctaccaacctaactgatgagtttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccagagttaccaagctgctccagggtgtgtcagccgcctccagaaatcctgcatggtgagcataccccaagccatcaggacaacttttcacctgggcaggaagtgttctacagctgtgagcctggctatgacctcagaggggctgcgtctctgcactgcacaccccagggagactggagccctgaagccccgagatgtgcagtgaaatcctgtgatgacttcttgggtcaactccctcatggccgtgtgctatttccacttaatctccagcttggggcaaaggtgtcctttgtctgtgatgaagggtttcgcttaaagggcagttccgttagtcattgtgtcttggttggaatgagaagcctttggaataacagtgttcctgtgtgtgaacatatcttttgtccaaatcctccagctatccttaatgggagacacacaggaactccctctggagatattccctatggaaaagaaatatcttacacatgtgacccccacccagacagagggatgaccttcaacctcattggggagagcaccatccgctgcacaagtgaccctcatgggaatggggtttggagcagccctgcccctcgctgtgaactttctgttcgtgctggtcactgtaaaaccccagagcagtttccatttgccagtcctacgatcccaattaatgactttgagtttccagtcgggacatctttgaattatgaatgccgtcctgggtattttgggaaaatgttctctatctcctgcctagaaaacttggtctggtcaagtgttgaagacaactgtagacgaaaatcatgtggacctccaccagaacccttcaatggaatggtgcatataaacacagatacacagtttggatcaacagttaattattcttgtaatgaagggtttcgactcattggttccccatctactacttgtctcgtctcaggcaataatgtcacatgggataagaaggcacctatttgtgagatcatatcttgtgagccacctccaaccatatccaatggagacttctacagcaacaatagaacatcttttcacaatggaacggtggtaacttaccagtgccacactggaccagatggagaacagctgtttgagcttgtgggagaacggtcaatatattgcaccagcaaagatgatcaagttggtgtttggagcagccctccccctcggtgtatttctactaataaatgcacagctccagaagttgaaaatgcaattagagtaccaggaaacaggagtttcttttccctcactgagatcatcagatttagatgtcagcccgggtttgtcatggtagggtcccacactgtgcagtgccagaccaatggcagatgggggcccaagctgccacactgctccagggtgtgtcagccgcctccagaaatcctgcatggtgagcataccctaagccatcaggacaacttttcacctgggcaggaagtgttctacagctgtgagcccagctatgacctcagaggggctgcgtctctgcactgcacgccccagggagactggagccctgaagcccctagatgtacagtgaaatcctgtgatgacttcctgggccaactccctcatggccgtgtgctacttccacttaatctccagcttggggcaaaggtgtcctttgtttgcgatgaagggttccgattaaaaggcaggtctgctagtcattgtgtcttggctggaatgaaagccctttggaatagcagtgttccagtgtgtgaacaaatcttttgtccaaatcctccagctatccttaatgggagacacacaggaactccctttggagatattccctatggaaaagaaatatcttacgcatgcgacacccacccagacagagggatgaccttcaacctcattggggagagctccatccgctgcacaagtgaccctcaagggaatggggtttggagcagccctgcccctcgctgtgaactttctgttcctgctgcctgcccacatccacccaagatccaaaacgggcattacattggaggacacgtatctctatatcttcctgggatgacaatcagctacacttgtgaccccggctacctgttagtgggaaagggcttcattttctgtacagaccagggaatctggagccaattggatcattattgcaaagaagtaaattgtagcttcccactgtttatgaatggaatctcgaaggagttagaaatgaaaaaagtatatcactatggagattatgtgactttgaagtgtgaagatgggtatactctggaaggcagtccctggagccagtgccaggcggatgacagatgggaccctcctctggccaaatgtacctctcgtgcacatgatgctctcatagttggcactttatctggtacgatcttctttattttactcatcattttcctctcttggataattctaaagcacagaaaaggcaataatgcacatgaaaaccctaaagaagtggctatccatttacattctcaaggaggcagcagcgttcatccccgaactctgcaaacaaatgaagaaaatagcagggtccttcct

SEQ ID NO: 2 - protein, 1998 aa, Homo sapiens
  MISC_FEATURE (1)..(1998): Full Length Protein
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYTCDPGYLLVGKGFIFCTDQGIWSQLDHYCKEVNCSFPLFMNGISKELEMKKVYHYGDYVTLKCEDGYTLEGSPWSQCQADDRWDPPLAKCTSRAHDALIVGTLSGTIFFILLIIFLSWIILKHRKGNNAHENPKEVAIHLHSQGGSSVHPRTLQTNEENSRVLP

SEQ ID NO: 3 - protein, 1931 aa, Homo sapiens
  MISC_FEATURE (1)..(1931): soluble CR1 polypeptide
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRAHDA

(19) United States
(12) Patent Application Publication
(10) Pub. No.: US 2007/0004625 A1
(43) Pub. Date: Jan. 4, 2007

(54) Use of complement inhibitory proteins to treat spinal cord injury

(76) Inventors: Liang-man Li, Shenyang (CN); Yue Zhu, Shenyang (CN); Guang-yu Fan, Shenyang (CN)

Correspondence Address: LEON R. YANKWICH;YANKWICH & ASSOCIATES, 201 BROADWAY, CAMBRIDGE, MA, 02139, US

(21) Appl. No.: 11/173,310

(22) Filed: Jun. 30, 2005

(51) Int. Cl. A61K 38/17

(52) U.S. Cl. 514/12

(57) ABSTRACT

Trauma to the spinal cord initiates an inflammatory response that results in secondary injury to the surrounding tissue, thereby exacerbating the effects of the initial injury. These secondary injury effects are contributed to, in part, by the activation of complement and the associated inflammatory reaction at the site of injury. The present invention describes a method for treating and/or ameliorating these secondary effects and improving locomotor function of a vertebrate that has suffered a spinal cord injury by administering a complement inhibitory protein to the individual as soon as possible after the initial injury occurs. According to this method, by inhibiting the activation of complement, inflammation and the resulting secondary tissue injury are reduced, thereby improving the prognosis of a patient suffering a spinal cord injury.

### FIELD OF THE INVENTION

The present invention relates to the use of complement inhibitory proteins, in particular soluble complement receptor type I (sCR1), to treat spinal cord injury.

### BACKGROUND OF THE INVENTION

It is estimated that 7800 people in the United States suffer spinal cord injuries (SCI) each year and that approximately 250,000 currently suffer from some form of spinal cord injury or spinal dysfunction. (National Spinal Cord Injury Association). Currently there is no cure for spinal cord injury.

SCI is a condition characterized by contusion of the neural tissue with resultant decrease or loss of its ability to function properly and transmit nerve impulses. It has been shown that all neural damage after an SCI event does not occur instantaneously. Following the initial injury, presumably as part of the immune response to the injury, a series of degenerative processes which promote tissue damage beyond the original site of injury are initiated. After the initial mechanical disruption of nerves and nerve fibers at the time of injury, hemorrhaging is usually observed within 30 minutes at the area of damage and may expand over the next few hours. Within several hours following the injury, inflammatory cells, e.g., neutrophils and macrophages, infiltrate the area and cause further damage to the nerve tissue, i.e., cell-mediated damage. These post-traumatic events are referred to as “secondary injury”.

It would be advantageous to prevent further damage to the spinal cord and surrounding tissue following a spinal cord injury by treatment as soon as possible after the initial trauma to prevent secondary injury effects.

Currently, the conventional treatment for reducing or minimizing the damage resulting from secondary injury is intravenous injection of the glucocorticoid, methylprednisolone (Bracken et al., JAMA, 277(20): 1597-1604 (1997). Unfortunately, prolonged administration of glucocorticoids has adverse systemic side effects, e.g., increased incidence of sepsis and pneumonia, and a limited therapeutic window.

Recently, Stokes et al. reported improved motor function in Lewis rats having a contusion type injury to the spinal cord after injection with liposomes containing dichloromethylene diphosphonate (“Cl₂MBP”) (U.S. Pat. No. 5,932,563). In addition, progress has been reported in improving motor function after spinal cord injury by treatment with α-lipoic acid and/or dihydrolipoic acid (DHL) (U.S. Pat. No. 6,432,434), α_{d} monoclonal antibodies (U.S. Pat. No. 6,432,404), and monosialoganglioside (GM₁) (U.S. Pat. No. 6,620,793).

Neurons and oligodendrocytes may be particularly vulnerable to complement-mediated cell death (Gasque et al., 1995, Journal of Immunology, 154(9): 4726-4733; Agoropoulou et al., 1998, Neuroreport, 9(5): 927-932; Wren et al., 1989, Proc. Natl. Acad. Sci. U.S.A., 86: 9025-9029), suggesting that complement activation can exacerbate damage in the injured CNS by contributing to demyelination and neurodegeneration. Additionally, cell receptor internalization of C5a can also induce apoptosis (Farkas et al., 1998, Neuroscience, 86(3): 903-911; Farkas et al., 1998, J. Physiology, 507(3): 679-687).

Constituting about 10% of the globulins in normal serum, the complement system is composed of many different proteins that are important in the immune system's response to foreign antigens. The complement system becomes activated when its primary components are fragmented and the fragments, alone or with other proteins, activate additional complement proteins resulting in a proteolytic cascade. Activation of the complement system leads to increased vascular permeability, chemotaxis of phagocytic cells, activation of inflammatory cells, opsonization of foreign particles, direct killing of cells and, as seen with spinal cord secondary injury, tissue damage. In view of the continuing need for effective treatments for spinal cord injury, new approaches to addressing post-traumatic phenomena are desirable.

### SUMMARY OF THE INVENTION

We have discovered that administration of a complement inhibitory protein, in particular soluble complement receptor type I, or sCR1, after spinal cord injury, is a surprisingly effective method for inhibiting inflammation and ameliorating the secondary injury effects described above which are associated with spinal cord injury. Soluble CR1 treatment is effective in improving motor function following traumatic spinal cord injury, as demonstrated herein in vivo utilizing a rat spinal cord injury model.

Accordingly, in its broadest aspects, the present invention relates to a method for treating spinal cord injury in a vertebrate subject comprising administration of a complement inhibitory protein following spinal cord injury. The treatment is effective to ameliorate or inhibit the damaging effects of secondary injury at the trauma site and to improve motor function. The complement inhibitory protein is preferably administered as soon as possible following a spinal cord injury to prevent the damaging effects of complement activation occurring at the site of injury. In preferred embodiments, the complement inhibitory protein is a soluble complement receptor type I (sCR1), i.e., a truncated, non-membrane-bound fragment of complement receptor type I, which fragment retains the complement regulatory properties of the natural or full-length CR1 protein, in particular the ability to inhibit complement activation and/or the ability to bind complement protein C3b or C4b, or (preferably) both C3b and C4b.

Furthermore, the present invention is directed to a method for ameliorating or inhibiting the secondary injury effects following a spinal cord injury in a vertebrate, which effects are associated with complement activation at the site of the injury. Preferably, said treatment comprises administration of a complement inhibitory protein as soon as possible after the initial injury. Preferably, the complement inhibitory protein is a soluble CR1 comprising at least the N-terminal two short consensus repeats (SCRs) of CR1, more preferably the extracellular domain of full-length human CR1, e.g., comprising amino acids 1-1930 of mature human CR1, most preferably the sCR1 polypeptide having the amino acid sequence of SEQ ID NO:3 (TP10; AVANT Immunotherapeutics, Inc., Needham, Mass. (USA)). Preferably the complement inhibitory protein is administered in a pharmaceutically acceptable carrier.

In yet another aspect, the present invention provides a method for improving locomotor function in a vertebrate that has suffered a spinal cord injury, the method comprising administration of a complement inhibitory protein after the initial trauma, e.g., to reduce the secondary injury effects caused by complement activation and the resulting inflammatory reaction which leads to further damage to the tissue surrounding the spinal cord. In preferred embodiments, the complement inhibitory protein is a soluble CR1 comprising at least the N-terminal two short consensus repeats (SCRs) of CR1, more preferably the extracellular domain of full-length human CR1, e.g., comprising amino acids 1-1930 of mature human CR1, most preferably the sCR1 polypeptide having the amino acid sequence of SEQ ID NO:3 (TP10; AVANT Immunotherapeutics, Inc.). Preferably, said sCR1 is administered in a pharmaceutically acceptable carrier.

In another aspect, the present invention provides a pharmaceutical composition suitable for inhibiting secondary injury following a spinal cord injury, said composition comprising a complement inhibitory molecule suitable for inhibiting complement activation and the secondary tissue damage at the site of injury resulting from post-traumatic complement activation. In preferred embodiments, the complement inhibitory protein is a soluble CR1 comprising at least the N-terminal two short consensus repeats (SCRs) of CR1, more preferably the extracellular domain of full-length human CR1, e.g., comprising amino acids 1-1930 of mature human CR1, most preferably the sCR1 polypeptide having the amino acid sequence of SEQ ID NO:3.

In yet another aspect, the present invention provides a method for preventing neutrophil activation and neutrophil infiltration of tissue as a result of complement activation at the site of spinal cord injury by administration of a complement inhibitory protein. In preferred embodiments, the complement inhibitory protein is a soluble CR1 comprising at least the N-terminal two short consensus repeats (SCRs) of CR1, more preferably the extracellular domain of full-length human CR1, e.g., comprising amino acids 1-1930 of mature human CR1, most preferably the sCR1 polypeptide having the amino acid sequence of SEQ ID NO:3.

In a particularly preferred embodiment, it is envisioned that the present invention will be suitable for treatment of humans suffering a spinal cord injury by systemic administration of sCR1 as soon as possible after the injury occurs.

The method of this invention can be practiced by using any complement inhibitory protein, such as, for example CR1, factor H, C4-binding protein (C4-BP), membrane cofactor protein (MCP), decay accelerating factor (DAF), or fragments thereof that retain complement inhibiting properties. Alternatively, the complement inhibitory protein may be an antibody specific for a complement protein, an activated complement protein, or a fragment of a complement protein, such antibody being useful to inhibit complement activation (e.g., anti-C3, anti-C5b-9, and the like). However, in preferred embodiments of the present invention, the complement inhibitory protein is human CR1, more preferably a soluble CR1 (sCR1), and most preferably the CR1 polypeptide comprising the extracellular domain of mature human CR1 or the soluble CR1 polypeptide having the amino acid sequence of SEQ ID NO:3.

### DETAILED DESCRIPTION

The present invention is directed to compositions and methods for treating spinal cord injury. In particular, the present invention is directed to compositions and methods for inhibiting the secondary effects known to exacerbate the initial injury and known to result, at least in part, from complement activation. In particular, the present invention is directed to a method for treating spinal cord injury comprising administration of a complement inhibitory protein as soon as possible after the initial spinal cord injury to ameliorate or inhibit the secondary injury effects that occur as a result of the initial trauma. After an initial traumatic event that results in a spinal cord injury (SCI), an inflammatory response at the site of injury results in further damage to the surrounding tissue, thereby exacerbating the effects of the injury and prolonging or preventing recovery. According to the present invention, administration of a complement inhibitory protein such as sCR1, as soon as possible after the initial injury, inhibits or diminishes the subsequent complement-mediated inflammatory response which in turn ameliorates the severity and effects of secondary injury. The treatment described herein can improve locomotor function over time in subjects suffering from a traumatic spinal cord injury.

The method of this invention can be practiced by using any complement inhibitory protein which is effective to block complement activation. Such complement inhibitory proteins include, for example, complement receptor type I (CR1), factor H, C4-binding protein (C4-BP), membrane cofactor protein (MCP), decay accelerating factor (DAF), or fragments thereof that retain complement inhibiting properties, such as the ability to inhibit complement activation, to bind C3b, to bind C4b, or to bind both C3b and C4b. Alternatively, the complement inhibitory protein may be an antibody specific for a complement protein, an activated complement protein, or a fragment of a complement protein, such antibody being effective to inhibit complement activation (e.g., anti-C3, anti-C5b-9, and the like; collectively refered to as “complement-inhibiting antibodies”). Preferably, the complement inhibitory protein used in the methods described herein is a soluble (non-membrane-bound) form of human CR1. Suitable soluble CR1 polypeptides and preparations are described in detail, e.g., in U.S. Pat. No. 5,981,481; U.S. Pat. No. 5,456,909; and U.S. Pat. No. 6,193,979. Special mention is made of a soluble CR1 polypeptide having glycosylation modified to exhibit sialyl Lewis X moieties (sCR1-sLe^{x}), as described in U.S. Pat. No. 6,193,979. More preferably, the method of the invention utilizes a polypeptide comprising the extracellular domain of mature human CR1 (for full-length mature human CR1 sequence, see: SEQ ID NO: 2). Most preferably, the method of the invention and pharmaceutical compositions useful for practicing the invention will include a soluble human CR1 protein having the amino acid sequence of SEQ ID NO:3.

As discussed more fully below, it has been demonstrated herein that administration of sCR1 following spinal cord injury in a rat spinal cord injury model reduces the activity and level of neutrophil infiltration of tissue and reduces the level of expression of complement-related proteins at the injury site as well as improves the overall recovery, i.e., locomotor function in the sCR1-treated rats as compared to normal saline-treated controls. We have thus discovered that administration of a complement inhibitory protein following a spinal cord injury in a vertebrate subject reduces the secondary injury effects caused by complement activation after the initial spinal cord injury.

In a specific embodiment, the invention relates to soluble CR1 polypeptides and their use for the treatment of spinal cord injury. As used herein, the terms “soluble CR1 polypeptides” or “soluble CR1” or “sCR1” shall be used to refer to portions of the full-length CR1 protein which, in contrast to the native CR1 proteins, are not expressed on the cell surface as transmembrane proteins. In particular, CR1 polypeptides which substantially lack a transmembrane region, or, preferably, which comprise all or part of the extracellular portion of CR1, are soluble CR1 polypeptides. In a preferred embodiment, the soluble CR1 polypeptides useful in the present invention are secreted by a cell in which they are expressed.

The human C3b/C4b receptor, termed complement receptor type I (CR1) or CD35, is present on the membranes of erythrocytes, monocytes/macrophages, granulocytes, B cells, some T cells, splenic follicular dendritic cells, and glomerular podocytes. (Fearon D. T., 1980, J. Exp. Med., 152: 20, Wilson, J. G., et al., 1983, J. Immunol., 131: 684). CR1 specifically binds C3b, C4b, and iC3b.

CR1 can inhibit the classical and alternative pathway C3/C5 convertases and act as a cofactor for the cleavage of C3b and C4b by factor I, indicating that CR1 also has complement regulatory functions in addition to serving as a receptor. (Fearon, D. T., 1979, Proc. Natl. Acad. Sci. U.S.A., 76: 5867; Iida, K. I. and Nussenzweig, V., 1981, J. Exp. Med., 153: 1138). In the alternative pathway of complement activation, the bimolecular complex C3b-Bb is a C3 enzyme (convertase). CR1 can bind to C3b thereby promoting the dissociation of fragment Bb from the complex. Furthermore, binding of C3b to CR1 renders C3b susceptible to irreversible proteolytic inactivation by factor I, resulting in the production of inactivated derivatives of C3b (namely, iC3b, C3d and C3dg). In the classical pathway of complement activation, the complex C3bC4bC2a is the C5 convertase. CR1 binds to C4b and/or C3b thereby promoting the dissociation of C2a from the complex. The binding renders C4b and/or C3b susceptible to irreversible proteolytic inactivation by factor I.

Several soluble (non-membrane bound) fragments of CR1 have been generated via recombinant DNA procedures by eliminating the transmembrane and cytoplasmic regions from the DNAs being expressed. See, e.g., Fearon et al., Intl. Patent Publ. WO 89/09220, Oct. 5, 1989; Fearon et al., Intl. Patent Publ. WO 91/05047, Apr. 18, 1991). The soluble CR1 fragments are functionally active, i.e., retaining the ability to bind C3b and/or C4b, inhibiting complement activation, and demonstrating factor I cofactor activity, depending upon the native CR1 regions the CR1 fragments contain. Such constructs inhibit in vitro the consequences of complement activation such as neutrophil oxidative burst, complement mediated hemolysis, and C3a and C5a production. A soluble construct, sCR1/pBSCR1c, also has demonstrated in vivo activity in a reversed passive Arthus reaction (Fearon et al., 1989, 1991, supra; Yeh et al., 1991, J. Immunol., 146:250 (1991), suppressed post-ischemic myocardial inflammation and necrosis (Fearon et al., supra; Weisman et al., 1990, Science, 249: 146-151) and extended survival rates following transplantation (Pruitt et al., 1991, J. Surg. Res., 50: 350; Pruitt et al., 1991, Transplantation, 52: 868 (1991)).

The complete cDNA coding sequence of the human CR1 protein is shown in SEQ ID NO:1. The amino acid sequence of mature human CR1 is shown in SEQ ID NO:2.

The isolation of the full-length CR1 gene, expression and purification of the full-length protein and active fragments thereof, and demonstration of activity in the full-length protein and fragments derived from the full-length protein, are described in U.S. Pat. No. 5,981,481, which is incorporated herein by reference.

The complement inhibitory proteins such as sCR1 that are useful in the methods of the present invention are advantageously produced in quantity using recombinant DNA technology to express the protein in a host cell, such as bacterial cells, mammalian cells, or even plant cells. For the complement inhibitory proteins contemplated herein, mammalian host cells, such as Chinese Hamster ovary (CHO) or African Green Monkey kidney (COS) cells, are preferred. The isolated gene encoding the desired protein can be inserted into an appropriate cloning vector. A large number of vector-host systems known in the art may be used. Possible vectors include, but are not limited to, plasmids or modified viruses. The vector system must be compatible with the host cell used. Such vectors include, but are not limited to, bacteriophages such as lambda derivatives, or plasmids such as pBR322 or pUC plasmid or CDM8 plasmid (See, B., 1987, Nature, 329: 840-842) or derivatives of those well-known vectors. Recombinant molecules can be introduced into host cells via transformation, transfection, infection, electroporation, etc.

Recombinant cells producing a preferred form of sCR1 are available from the American Type Culture Collection, Rockville, Md. (accession no. CRL 10052). The deposited cells are a Chinese Hamster ovary cell line DUX B11 carrying plasmid pBSCR1c/pTCSgpt clone 35.6, encoding a soluble CR1 having the amino acid sequence of SEQ ID NO:3. Such sCR1 protein in purified form is produced under the product designation TP10 by AVANT Immunotherapeutics, Inc. (Needham, Mass.).

After expression in a host cell, the soluble CR1 molecules may be isolated and purified by standard methods including chromatography (e.g., ion exchange, affinity, and sizing column chromatography, high pressure liquid chromatography), centrifugation, differential solubility, or by any other standard technique for the purification of proteins. Preferred purification methods are described in U.S. Pat. No. 6,316,604, U.S. Pat. No. 5,252,216, and U.S. Pat. No. 5,840,858.

Soluble CR1 proteins are therapeutically useful in the modulation of complement-mediated diseases, that is, diseases or conditions characterized by inappropriate or undesired complement activation. A soluble CR1 protein or fragment which can bind C3b or C4b, and/or retain the ability to inhibit the alternative or classical C3 or C5 convertases, and/or retain factor I cofactor activity, can be used to inhibit complement activation. In the present invention, we have demonstrated that soluble CR1 can be used to ameliorate or inhibit undesirable complement activity following traumatic spinal cord injury, e.g., post-traumatic inflammatory conditions resulting from complement activation.

In the method of the invention, a complement inhibitory protein, such as soluble CR1, is administered, preferably intravenously, to a vertebrate subject who has suffered spinal cord injury in order to attenuate complement activation, to reduce or inhibit inflammation associated with the spinal cord injury and to reduce or inhibit detrimental secondary injury effects associated with such inflammation.

In a method of treating spinal cord injury according to the invention, a therapeutically active amount of a complement inhibitory protein or preparation thereof is administered to a subject in need of such treatment. The preferred subject is a human.

The amount administered should be sufficient to inhibit complement activation or inhibit the secondary effects of acute inflammation that follow spinal cord injury, such as reducing neutrophil infiltration to injured tissues or reducing neutrophil activity. (See, Example 3, supra.) The determination of a therapeutically effective dose is within the capability of practitioners in this art, however, as an example, in embodiments of the method described herein utilizing sCR1 for the treatment of spinal cord injury, an effective dose will be in the range of 0.01-100 mg/kg; preferably 0.1-10 mg/kg, most preferably 1-10 mg/kg patient body weight. The pharmaceutical composition should be administered as soon as possible after the spinal cord injury, and repeated doses are contemplated in order to maintain an effective amount, e.g., to reduce or inhibit complement activation. For example, as demonstrated in the examples below, and effective regimen of sCR1 administration consisted of a single injection (6 mg/kg) of sCR1 on the day of spinal cord injury, followed with a similar dose by intravenous injection every day thereafter for the course of treatment.

For administration, the sCR1 or other therapeutic protein may be formulated into an appropriate pharmaceutical composition. Such a composition typically contains a therapeutically active amount of the sCR1 or other protein and a pharmaceutically acceptable excipient or carrier such as saline, buffered saline, phosphate buffers, dextrose, or sterile water. Compositions may also comprise specific stabilizing agents such as sugars, including mannose and mannitol.

Various delivery systems are known and can be used for delivery of CR1 and soluble fragments of CR1, e.g., encapsulation in liposomes, microparticles, or microcapsules. Suitable modes of administration include but are not limited to, intradermal, intramuscular, intraperitoneal, intravenous, subcutaneous, intrathecal, or epidural injection, and oral or pulmonary delivery.

In a preferred embodiment, pharmaceutical compositions for use in the present invention may be formulated in accordance with routine procedures as a pharmaceutical composition for intravenous administration to an individual suffering a spinal cord injury. Typically compositions for intravenous administration are solutions in sterile aqueous buffer. Where necessary, the composition may also include a solubilizing agent and a local anesthetic such as lidocaine to ease pain at the site of injection. Generally, the ingredients will be supplied either separately or mixed together in unit dosage form, for example, as a dry lyophilized powder or water free concentrate in a hermetically sealed container such as an ampoule or sachette indicating the quantity of active agent in activity units. Where the composition is to be administered by injection, an ampoule of sterile water for injection or saline may be provided so that the ingredients may be mixed prior to administration.

A pharmaceutical pack comprising one or more containers filled with one or more of the ingredients of the pharmaceutical composition is also contemplated. This is particularly advantageous as it is perceived that administration of the complement inhibitory protein according to the method of the present invention for the treatment of spinal cord injury could be advantageously administered by emergency personnel on site, e.g., at the site of an automobile accident resulting in a spinal cord injury, as it is critical that the complement inhibitory protein be administered to an individual suffering a spinal cord injury as soon as possible after injury, preferably immediately or within hours or minutes after the initial spinal cord injury occurs.

### EXAMPLES

The following examples illustrate the methods of the present invention. They are provided by way of illustration and not for purposes of limitation.

All data were presented as mean value±standard deviation (x±s). Using SPSS 10.0 statistical analysis software, homogeneity tests for variance were first performed, according to different timepoints, between the treatment and control groups, then t tests were performed.

### Example 1

To test whether administration of a complement inhibitory protein can ameliorate or inhibit secondary injury following a traumatic event involving the spinal cord and improve locomotor function over time, Sprague-Dawley (SD) rats (n=80) all between 250 g-300 g (Experimental Animal Department of China Medical University) were administered a spinal cord injury by a modified Allen's striking method (Black et al., 1988, Neuro. Surg., 22:51-60) using a 2 mm diameter concave plastic pad placed over the target site (T₁₀ segment) and a striking force of 50 g/cm.

After injury, the 80 rats were divided randomly into 10 separate groups of 8 rats/group. Five of the 10 groups (n=40) were comprised of rats receiving sCR1 (TP10, AVANT Immunotherapeutics, Inc.) injections, and the remaining 5 groups (n=40) were comprised of control rats receiving normal saline injections only. Each of the sCR1 groups and each of the normal saline groups was assigned a separate evaluation time point, i.e., 12 hours, 1 day, 3 days, 7 days, and 14 days following the initial spinal cord injury, respectively. All rats receiving injections of sCR1 (n=40) were administered caudal intravenous injections of 6 mg/kg at 1 hour after the initial injury, followed by 1 injection/day (6 mg/kg) for the duration of the experiment. Control rats (n=40) received caudal intravenous injections (6 mg/kg) of normal saline at 1 hour after initial injury followed by 1 injection/day (6 mg/kg) for the duration of the experiment.

Nerve Function Evaluation

Recovery of locomotor function of the lower extremities over time was tested using a tilt board method. The rats were placed on the same smooth board at 12 hours, 1 day, 3 days, 7 days, and 14 days after injury. The body axis of the rats was aligned with the longitudinal axis of the tilt board. The longitudinal angle of the tilt board was raised in increments of 5°, and locomotor recovery was evaluated as a function of the maximum tilt angle at which the rat could remain in position on the board. Each rat was tested three times and the mean angle recorded. The results of the tilt board experiment are shown in Table 1.

TABLE 1
Tilt board experiment (mean angle, x ± s)

| Timepoint | | Control | receiving | | |
|---|---|---|---|---|---|
| Group | | (receiving saline) | sCR1 | t | P |
| 12 | hrs | 33.81 ± 2.75 | 34.12 ± 2.34 | 0.21 | >0.05 |
| 1 | day | 34.97 ± 2.69 | 37.57 ± 3.41 | 1.46 | >0.05 |
| 3 | days | 36.32 ± 3.72 | 40.91 ± 3.66 | 2.15 | <0.05 |
| 7 | days | 38.59 ± 3.12 | 43.24 ± 2.81 | 2.71 | <0.01 |
| 14 | days | 44.25 ± 3.78 | 51.58 ± 3.62 | 3.43 | <0.01 |

As seen in Table 1, there was no significant difference in the angle at which sCR1-treated and normal saline-treated (control) rats were able to remain on the tilt board at 12 hours and 1 day after injury; however, the locomotor function of sCR1-treated rats at 3 days, 7 days, and 14 days after treatment was significantly better than the normal saline-treated controls. The results in Table 1 demonstrate that administration of sCR1 following spinal cord injury inhibits the nerve damage at the site of injury, resulting in an improvement in recovery of motor function.

### Example 2

Pathohistological Examination

Three rats from each timepoint group were anesthetized at their respective time points (12 hr, 1 day, 3 days, 7 days, and 14 days after injury), and 1 cm of spinal tissue from the injury site was taken, fixed and dehydrated. 12 μm thick serial sections were made using a freezing microtome and prepared for hematoxylin-eosin (HE) staining or C3c, C9 or CD59 immunohistochemical staining. Rabbit anti-rat C3c antibody was purchased from Zymed Laboratories (Invitrogen Corp., Carlsbad, Calif.). Rabbit anti-rat C9 and rabbit anti-CD59 antibodies were obtained from Prof. Morgan (Wales University, U.K.). HE staining kits were purchased from Sigma-Aldrich Biotechnology Center (SABC; Sigma-Aldrich Corp., St. Louis, Mo.).

Results: HE Staining

The results of HE staining were observed microscopically (200×). In the normal saline control group rats, at 12 hrs after injury, there was spotty hemorrhaging in the gray matter and neutrophils began to infiltrate the tissue around the injury site. At 1 day after injury in the normal saline group, neurons were swelling and exhibited dissymmetry of the nuclei. At 3 days after injury, the neurons of the normal saline rats became obviously rounded and swollen, and nuclear pyknosis (indicating apoptosis) and nuclear fragmentation could be seen. In addition, there was a significant amount of neutrophil infiltration of the tissue from the injury site and large spotty hemorrhaging in the gray matter. At 7 days after injury, the remnant neurons decreased in number but some neutrophil infiltration still existed in the gray matter. At 14 days after injury, fewer remnant neurons remained, and a lesser amount of neutrophil infiltration of the gray matter was observed. Porosis could be seen.

In both the sCR1 and control groups, the injured spinal cord deteriorated over time, reaching a peak around day 3 after injury and tending to be stabilized by day 14 after injury. However, in the sCR1-treated groups, all the symptoms associated with secondary effects at the site of injury, i.e., neuron cellular swelling, degeneration, and neutrophil infiltration were notably less severe than in the control group rats as described above.

Results: C3c Immunohistochemical Staining

The presence of C3c at the site of injury indicates that complement activation is occurring at the site. The results of the C3c immunohistochemical staining demonstrated that C3c expression levels were lower at the site of injury in the sCR1-treated rats than in the normal saline-treated controls.

Three C3c immunohistochemical staining sections were selected from each rat in every group for analysis. Five high power fields were chosen from the anterior horn to the posterior horn of the gray matter. Average gray value (AG) of C3c-positive reactants was detected using a MetaMorph® automatic colored image analyzer (Universal Imaging Corp., Downington, Pa.). The average gray value is inversely related to C3c immune reaction intensity. The results of the C3c immunohistochemical staining are shown in Table 2.

TABLE 2
Image analysis results of C3c positive reactance in
injury tissue (AG, x ± s)

| Timepoint | | Control | | | |
|---|---|---|---|---|---|
| Group | | (receiving saline) | receiving sCR1 | t | P |
| 12 | hrs | 54.21 ± 3.08 | 59.37 ± 4.85 | 3.06 | <0.01 |
| 1 | day | 48.82 ± 4.75 | 56.25 ± 5.97 | 3.27 | <0.01 |
| 3 | days | 39.07 ± 2.26 | 43.81 ± 3.64 | 3.71 | <0.01 |
| 7 | days | 47.93 ± 3.99 | 55.20 ± 4.17 | 4.23 | <0.01 |
| 14 | days | 58.14 ± 4.72 | 66.23 ± 5.08 | 3.91 | <0.01 |

At 12 hrs after injury, in both the sCR1-treated and normal saline-treated rats, scattered C3c positive expression was observed in some neuron cytomembrane and cytoplasm in the spinal cord anterior horn. At 1 day after injury, C3c expression increased in both groups. At 3 days after injury, high levels of C3c-positive expression were apparent in the remnant neuron cytomembrane, neuropilem, and the neuron cytoplasm. At 7 days after injury, C3c expression decreased gradually. At day 14 after injury, some C3c-positive neurons were still present in the gray matter, and low levels of C3c expression were evident in the neuropilem.

In both the sCR1-treated and control groups, C3c-positive expression in spinal cord injury tissue peaked around day 3 after injury, began to decrease thereafter and was stable at 2 weeks after the injury. However, as seen in Table 2, C3c-positive expression levels were significantly less in the sCR1-treated rats at every timepoint, compared with the normal saline-treated rats (P<0.01).

Results: C9 and CD59 Immunohistochemical Staining

Data from C9 and CD59 immunohistochemical staining were obtained and observed by light microscope (200×) using the same techniques as used in detecting C3c expression.

C3 and C9 are important inherent components of the complement system. C3 is at the confluence of the classical and alternative complement activation pathways and acts as a hub in the complement activation cascade. The existence of C3c, which is a decomposition product of C3 after its inactivation, indicates that activation of the complement system has proceeded to the C3 level. C9, the last molecule that constitutes the membrane attack complex (MAC), is also the most important complement component for activation of the complement system that attacks and destroys target cells. The anti-C9 antibody employed herein was specific for C9 deposited in tissue, which is C9 integrated in the MAC. Therefore, the detection of C9 thoroughly reflected the MAC expression condition.

As with C3c, it was found that C9 was present in the spinal injury tissue at each timepoint after injury in both the sCR1 and the saline (control) groups, which indicates that the complement cascade reaction could be aroused and activated to the end phase in acute spinal cord injury.

The presence of C9 detected in the experiment was similar to that observed in the C3c assays described above. Image analysis results of the C9 detection assays are presented in Table 3.

TABLE 3
Image analysis results of C9 positive reactants
in injury tissue (AG, x ± s)

| Timepoint | | Control | | |
|---|---|---|---|---|
| Group | | (receiving saline) | receiving sCR1 | P |
| 12 | hrs | 82.18 ± 4.19 | 88.52 ± 5.03 | <0.01 |
| 1 | day | 73.46 ± 3.33 | 81.92 ± 2.24 | <0.01 |
| 3 | days | 61.75 ± 2.39 | 71.32 ± 2.32 | <0.01 |
| 7 | days | 47.93 ± 5.31 | 81.23 ± 5.30 | <0.01 |
| 14 | days | 85.82 ± 5.36 | 95.37 ± 3.06 | <0.01 |

As can be seen from the data of Table 3, the C9 expression in the sCR1-treated groups was significantly less than observed in the saline-treated groups (P<0.01).

CD59 is a homogeneous restriction factor in the complement end phase. It is an important protective complement regulatory factor, as its main biological activity is to inhibit the continued formation of MAC and to prevent cytolysis.

As with C3c and C9, it was found that CD59 was present in the spinal injury tissue at each timepoint after injury in both the sCR1 and the saline (control) groups, which indicates that the complement cascade reaction could be aroused and activated to the end phase in acute spinal cord injury.

The presence of CD59 detected in the experiment was similar to that observed in the C3c and C9 assays described above. Image analysis results of the CD59 detection assays are presented in Table 4.

TABLE 4
Image analysis results of C9 positive reactants in
injury tissue (AG, x ± s)

| Timepoint | | Control | | |
|---|---|---|---|---|
| Group | | (receiving saline) | receiving sCR1 | P |
| 12 | hrs | 95.12 ± 4.89 | 121.31 ± 9.62 | <0.01 |
| 1 | day | 87.46 ± 6.34 | 98.54 ± 8.09 | <0.01 |
| 3 | days | 69.08 ± 6.18 | 80.01 ± 5.43 | <0.01 |
| 7 | days | 80.36 ± 5.01 | 83.57 ± 6.10 | <0.05 |
| 14 | days | 96.46 ± 6.97 | 97.35 ± 7.94 | |

As seen in Table 4, the sCR1-treated groups at 12 hours, 1 day, 3 days and 7 days after injury showed a significant difference from the saline-treated controls, indicating that sCR1 was inhibiting complement activation. By day 14, the difference in CD59 expression was no longer significant, which was probably an indication that prolonged treatment with sCR1 was having the protective effect in the spinal injury tissue of reducing destruction of CD59 expressing cells (e.g., neurons, neuroglial cells)

Swelling, degeneration and necrosis of injured neurons were milder at all timepoints in the sCR1-treated groups. Also, less neutrophil infiltration of injured tissue was observed for samples taken from the sCR1-treated groups.

### Example 3

Myeloperoxidase Activity

Myeloperoxidase (MPO) is a marker enzyme for neutrophils, which are the main inflammatory cells involved in acute inflammatory reactions. Myeloperoxidase catalyzes the conversion of hydrogen peroxide (H₂O₂) and chloride anion (Cl⁻) to hypochlorous acid (HOCl). Hypochlorous acid is cytotoxic and is produced by neutrophils to destroy invading bacteria and other pathogens. The presence and levels of myeloperoxidase at the site of injury is thus a direct indication of the activity and level of neutrophil infiltration at the site.

To measure the levels of myeloperoxidase activity at the site of injury, 5 rats from each group at each timepoint, i.e., 12 hours, 1 day, 3 days, 7 days, and 14 days after injury, were anesthesized and a 1 cm sample of tissue from the spinal cord injury site was collected. The tissue was preserved in liquid nitrogen and sheared after weighing.

Tribasic potassium phosphate buffer solution with 0.5% cetrimonium bromide (CTBA) was added prior to homogenization. Each sample was then comminuted by ultrasound for 20 minutes at 4° C. then centrifuged at 12,500 rpm for 30 minutes. The supernatant was removed and added to 2.9 ml phosphate buffer containing 0.5% dianisidine hydrochloric acid and 0.0005% hydrogen peroxide (pH 7.0). Myeloperoxidase levels were measured spectrophotometrically at 460 nm over 2 minutes. One unit of myeloperoxidase activity was defined as the degradation of 1 μmol of hydrogen peroxide per minute at 25° C. Myeloperoxidase levels are expressed as units of myeloperoxidase activity per gram of spinal cord tissue.

The results of the myeloperoxidase assay are shown in Table 5.

TABLE 5
Myeloperoxidase activity (U/g wet weight, x ± s)

| Time | | NS group | sCR1 group | t | P |
|---|---|---|---|---|---|
| 12 | hrs | 0.896 ± 0.062 | 0.543 ± 0.053 | 4.84 | <0.01 |
| 1 | day | 1.271 ± 0.065 | 0.932 ± 0.049 | 4.65 | <0.01 |
| 3 | days | 1.805 ± 0.073 | 1.327 ± 0.069 | 5.32 | <0.01 |
| 7 | days | 1.125 ± 0.047 | 0.764 ± 0.051 | 5.82 | <0.01 |
| 14 | days | 0.714 ± 0.054 | 0.401 ± 0.047 | 4.89 | <0.01 |

The levels of myeloperoxidase in the sCR1-treated rats and the normal saline-treated rats began to increase at 12 hours after injury, reached peak levels 3 days after injury, and began to decrease thereafter. However, as seen in Table 5, the myeloperoxidase levels in the sCR1-treated groups were consistently lower at each timepoint than in the control groups (P<0.01). (See, Table 5.)

These results clearly demonstrate that administration of sCR1 following spinal cord injury reduces the levels and amount of neutrophil infiltration at the site of injury, thereby reducing inflammation and the secondary injury effects associated with inflammation.

The data of the foregoing examples demonstrate that administration of sCR1 following a spinal cord injury in a vertebrate inhibits the activation of complement and inhibits inflammation. Also, improved recovery of locomotor function after the injury was observed following treatment with sCR1. These data demonstrate the suitability of the methods disclosed herein for treating spinal cord injury in vertebrates, including humans.

Although a number of embodiments have been described above, it will be understood by those skilled in the art that modifications and variations of the described compositions and methods may be made without departing from either the disclosure of the invention or the scope of the appended claims. The articles and publications cited herein are incorporated by reference.

We claim:

1. A method for treating a spinal cord injury in a vertebrate subject comprising:
administering a complement inhibitory protein to said vertebrate as soon as possible after said injury.

2. The method according to claim 1, wherein said complement inhibitory protein is selected from the group consisting of: complement receptor type I (CR1), factor H, C4-binding protein (C4-BP), membrane cofactor protein (MCP), decay accelerating factor (DAF), fragments thereof that retain complement inhibiting properties, complement-inhibiting antibodies, and sCR1-sLe^{x}.

3. The method according to claim 2, wherein said vertebrate is a human.

4. The method according to claim 2, wherein said complement inhibitory protein is a soluble CR1 protein.

5. The method according to claim 4, wherein said soluble CR1 is a polypeptide comprising at least the N-terminal two short consensus repeats of full-length human CR1.

6. The method according to claim 5, wherein said soluble CR1 is a polypeptide comprising the extracellular domain of mature human CR1.

7. The method according to claim 4, wherein said soluble CR1 has the amino acid sequence of SEQ ID NO:3.

8. A method for improving the locomotor function of a vertebrate subject suffering from a spinal cord injury comprising: administering a complement inhibitory protein to said subject as soon as possible after said injury.

9. The method according to claim 8, wherein said complement inhibitory protein is selected from the group consisting of: complement receptor type I (CR1), factor H, C4-binding protein (C4-BP), membrane cofactor protein (MCP), decay accelerating factor (DAF), fragments thereof that retain complement inhibiting properties, complement-inhibiting antibodies, and sCR1-sLe^{x}.

10. The method according to claim 9, wherein said vertebrate is a human.

11. The method according to claim 9, wherein said complement inhibitory protein is a soluble CR1 protein.

12. The method according to claim 11, wherein said soluble CR1 is a polypeptide comprising at least the N-terminal two short consensus repeats of full-length human CR1.

13. The method according to claim 12, wherein said soluble CR1 is a polypeptide comprising the extracellular domain of mature human CR1.

14. The method according to claim 11, wherein said soluble CR1 has the amino acid sequence of SEQ ID NO:3.

15. A pharmaceutical composition for use in treating a spinal cord injury comprising a therapeutically effective amount of a soluble CR1 protein and a pharmaceutically acceptable excipient or carrier.

16. The pharmaceutical composition according to claim 15, wherein the soluble CR1 protein is a polypeptide comprising the extracellular domain of mature human CR1.

17. The pharmaceutical composition according to claim 15, wherein the soluble CR1 protein is a polypeptide having the amino acid sequence of SEQ ID NO:3.
